(19) United States
(12) Patent Application Publication
(10) Pub. No.: US 2024/0260860 A1
(43) Pub. Date: Aug. 8, 2024

(54) FLUID ANALYSIS USING A WEARABLE DEVICE

(71) Applicant: Oura Health Oy, Oulu (FI)

(72) Inventors: Jukka Tapani Mäkinen, Oulu (FI); Pauli Ohukainen, Oulu (FI); Olli Petteri Heikkinen, Oulu (FI); Heidi Similä, Oulu (FI); Mika Petteri Kangas, Oulu (FI); Matti Mäkiniemi, Oulu (FI); Erin Turkoglu, Oulu (FI); Teemu Juhani Haverinen, Oulu (FI); Sami Sakari Ihme, Oulu (FI)

(21) Appl. No.: 18/164,320

(22) Filed: Feb. 3, 2023

(51) Int. Cl. A61B 5/145; A61B 5/00; A61B 5/0205

(52) U.S. Cl. CPC A61B 5/14521; A61B 5/0075; A61B 5/0205; A61B 5/6826; A61B 5/6833; A61B 5/742

(57) ABSTRACT

Methods, systems, and devices for analyzing fluid using a wearable device are described. The method may include collecting a bodily fluid of a user using a fluid collection component that includes one or more measurement channels configured to change color when exposed to the bodily fluid. Further, the method may include transmitting light associated with one or more wavelengths and generating one or more signals based on the light reflected off the one or more measurement channels of the fluid collection component and received by a wearable device. Moreover, the method may include determining a color of the one or more measurement channels based on the one or more signals and determining a presence or concentration of one or more substances within the bodily fluid based on the color.

### FIELD OF TECHNOLOGY

The following relates to wearable devices and data processing, including fluid analysis using a wearable device.

### BACKGROUND

Some wearable devices may be configured to collect physiological data from a user and display a message associated with the physiological data such that the user may make informed decisions on their health. However, some wearable devices are limited with respect to how the wearable devices are able to measure physiological data, and what physiological parameters the wearable devices are able to measure. For example, bodily fluids (e.g., sweat) of a user may include components indicative of the user's health. For instance, a user's sweat may include salt. If the user consumes a large amount of salt (e.g., salt content of the sweat is above a threshold), the user may experience physiological problems (e.g., hypertension) or be at risk of the physiological problems. However, conventional wearable devices may include light-based devices that are unable to collect and/or accurately analyze such bodily fluids, thereby limiting the utility of such wearable devices.

### BRIEF DESCRIPTION OF THE DRAWINGS

FIG. 1 illustrates an example of a system that supports fluid analysis using a wearable device in accordance with aspects of the present disclosure.

FIG. 2 illustrates an example of a system that supports fluid analysis using a wearable device in accordance with aspects of the present disclosure.

FIG. 3 illustrates an example of a system that supports fluid analysis using a wearable device in accordance with aspects of the present disclosure.

FIG. 4 illustrates an example of a graphical user interface (GUI) that supports fluid analysis using a wearable device in accordance with aspects of the present disclosure.

FIG. 5 illustrates a block diagram of an apparatus that supports fluid analysis using a wearable device in accordance with aspects of the present disclosure.

FIG. 6 illustrates a block diagram of a wearable device manager that supports fluid analysis using a wearable device in accordance with aspects of the present disclosure.

FIG. 7 illustrates a diagram of a system including a device that supports fluid analysis using a wearable device in accordance with aspects of the present disclosure.

FIGS. 8 and 9 illustrate flowcharts showing methods that support fluid analysis using a wearable device in accordance with aspects of the present disclosure.

### DETAILED DESCRIPTION

A user of a wearable device may experience physiological health issues. In some examples, the physiological health issues may be a result of the user's diet. For example, if the user frequently consumes high amounts of salt (e.g., consumes an amount of salt above a threshold), the user may experience hypertension (e.g., increased blood pressure) which may lead to a heart attack or stroke. In some examples, electrolytes (e.g., sodium) circulating in the blood of the user may be included in a user's sweat. As such, a sodium content of the user's sweat may be indicative of the user's health. For example, a high concentration of sodium (e.g., concentration of sodium above a threshold) in the user's sweat may indicate that the user may be at risk of one or more physiological health issues, such as hypertension, heart attack, or stroke.

Some wearable devices may be configured to collect physiological data from a user and display a message associated with the physiological data such that the user may make informed decisions on their health. Such wearable devices may help the user identify potential health issues. However, some wearable devices are limited with respect to how the wearable devices are able to measure physiological data, and what physiological parameters the wearable devices are able to measure. For example, conventional wearable devices may include light-based devices that are unable to collect and/or accurately analyze such bodily fluids, thereby limiting the utility of such wearable devices.

Accordingly, aspects of the present disclosure are directed to techniques that utilize wearable devices to determine a presence and/or concentration of one or more substances within a user's bodily fluid. High levels of these substances (e.g., salt) may negatively impact the user's health. Moreover, the presence or concentration of other substances may provide the user with more insight regarding their overall health. As such, the ability of the wearable device to detect the presence or concentration of the substances and display such information to the user may allow the user to make informed decisions on their health and potentially avoid or mitigate physiological problems.

For example, in the context of a wearable ring device, a fluid collection component may be affixed to the user's finger. The fluid collection component may include an adhesive patch, a lateral flow patch, a microfluidic chamber, or the like. Further, the fluid collection component may include one or more measurement channels that are configured to change color when exposed to the user's bodily fluid (e.g., sweat, blood, saliva, or tears). The fluid collection component may collect bodily fluid from the user for some duration and subsequently, a wearable device affixed to the user's finger may transmit light along a signal path that travels through the one or more measurement channels. The wearable device may receive the light reflected off (and/or transmitted through) of the one or more measurement channels and generate a signal associated with the received light. The wearable device may determine a color of the measurement channel based on the signal and determine either a presence or a concentration of a substance included within the bodily fluid based on the color. In some examples, the wearable device may display a message associated with the presence or the concentration of the substance.

Aspects of the disclosure are initially described in the context of systems supporting physiological data collection from users via wearable devices. Additional aspects are described in the context of a graphical user interface (GUI). Aspects of the disclosure are further illustrated by and described with reference to apparatus diagrams, system diagrams, and flowcharts that relate to fluid analysis using a wearable device.

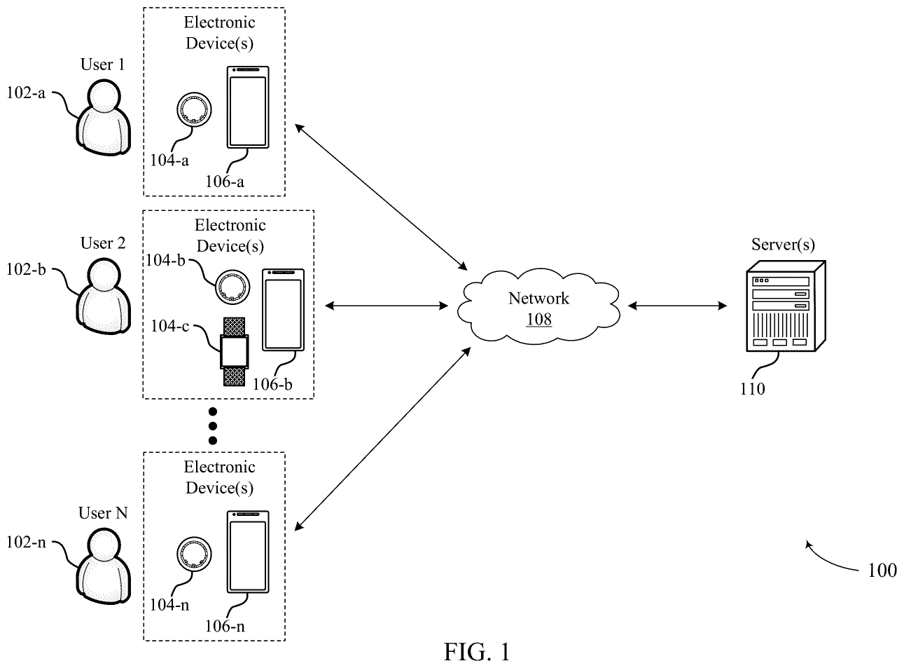

FIG. 1 illustrates an example of a system 100 that supports fluid analysis using a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a wearable device 104 to determine a presence or a concentration of a substance included in a user's bodily fluid. In some examples, a fluid collection component including one or more measurement channels may be placed on the user's finger such that the one or more measurement channels may be in contact with the palm-side of the user's finger. The fluid collection component may collect the bodily fluid from the user 102 and direct the bodily fluid to the one or more measurement channels (e.g., using microfluidic channels or using an absorbent pad). When exposed to the bodily fluid, the one or more measurement channels may change color and the color may be indicative of a presence or concentration of the substance included in the bodily fluid. For example, varying concentrations of salt within the user's sweat may cause the measurement channels to change to different colors and/or to different extents.

After collection of the bodily fluid, the wearable device 104 may transmit, using a light-emitting component proximate to the one or more measurement channels, a light associated with one or more wavelengths. The wearable device 104 may receive, using a photodetector proximate to the one or more measurement channels, light reflecting off of (or transmitted through) the one or more measurement channels and generate a signal indicating characteristics of the received light (e.g., signal strength of the received light). Using the signal, the wearable device 104 may determine the color of the one or more measurement channels and subsequently determine a presence or concentration of the substance included in the bodily fluid of the user 102 based on the determined color. In some examples, the wearable device 104 may transmit bodily fluid content data including the presence or concentration of the substance to a user device 106 associated with the wearable device 104. The user device 106 may process the received bodily fluid content data (e.g., compare the bodily fluid content data to past bodily fluid content data) and generate a message (e.g., reciting an increase or decrease in the substance) for display on a GUI of the user device 106.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

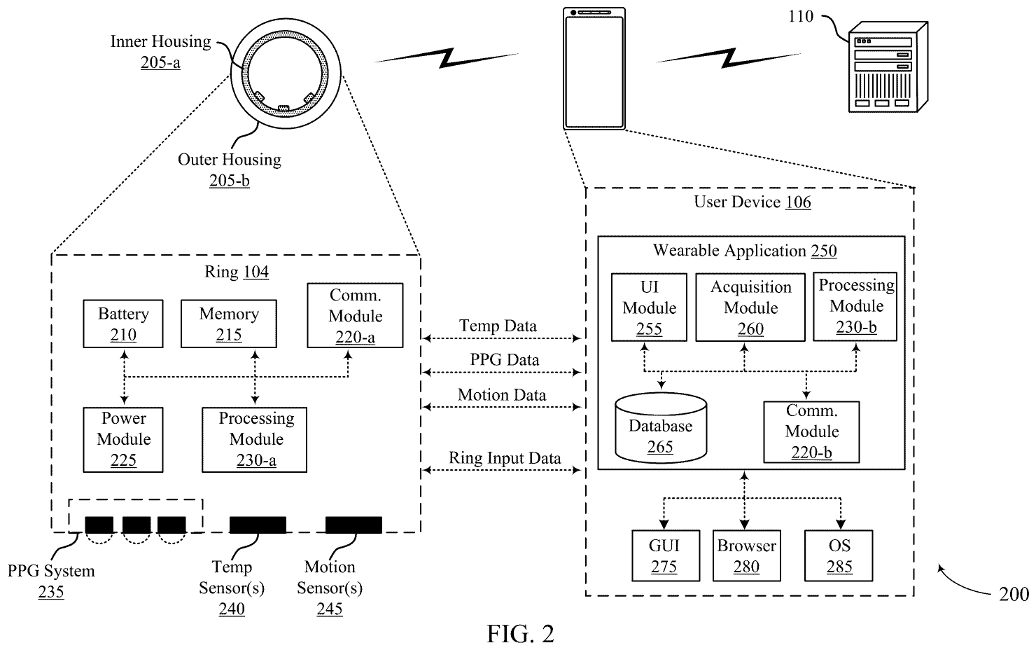

FIG. 2 illustrates an example of a system 200 that supports fluid analysis using a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist wom computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light-emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared (IR) spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for a ring 104 to determine a concentration or a presence of a substance included in the user's bodily fluid. In some examples, a fluid collection component such as an adhesive patch, a lateral flow patch, or a microfluidic chamber may be attached to a finger of the user or coupled to the inner-housing 205-a such that the fluid collection component is in contact with the finger of the user when the ring 104 is being worn. The fluid collection component may include at least one measurement channel. The measurement channel may include a material that changes color when in contact with one or more substances within a user's bodily fluid. The resulting color of the material may indicate the presence or lack thereof of the substance. Additionally or alternatively, the resulting color of the material may indicate a concentration of the substance. For a period of time (e.g., while the user is performing physical activity), the fluid collection component may collect bodily fluid from the user and direct the fluid to the measurement channel.

The ring 104 may be situated on top of the fluid collection component such that an optical transmitter of the ring 104 and an optical receiver of the ring 104 are proximate to the measurement channel. After collecting a bodily fluid from the user, the ring 104 may transmit, using the optical transmitter, one or more wavelengths of light and receive, using the optical receiver, the light from the optical transmitter. In some examples, the light may reflect off of the measurement channel to the optical receivers, or be transmitted through the measurement channel to the optical receivers. The ring 104 may analyze the received light and generate a signal indicating characteristics of the received light (e.g., signal strength of the received light) and the signal may be passed to the processing module 230-a. In some examples, the processing module 230-a may compare the received light with the transmitted light. For example, the processing module 230-a may compare the signal strength of the received light with the signal strength of the transmitted light to determine how much light was absorbed by the measurement channel (e.g., reflectance factor of the light). From this comparison, the processing module 230-a may determine the color of the measurement channel which may indicate a presence or concentration of the substance. For instance, the amount of light absorbed by the measurement channel may indicate the relative color of the measurement channel, and therefore the presence/concentration of substances collected within the measurement channel.

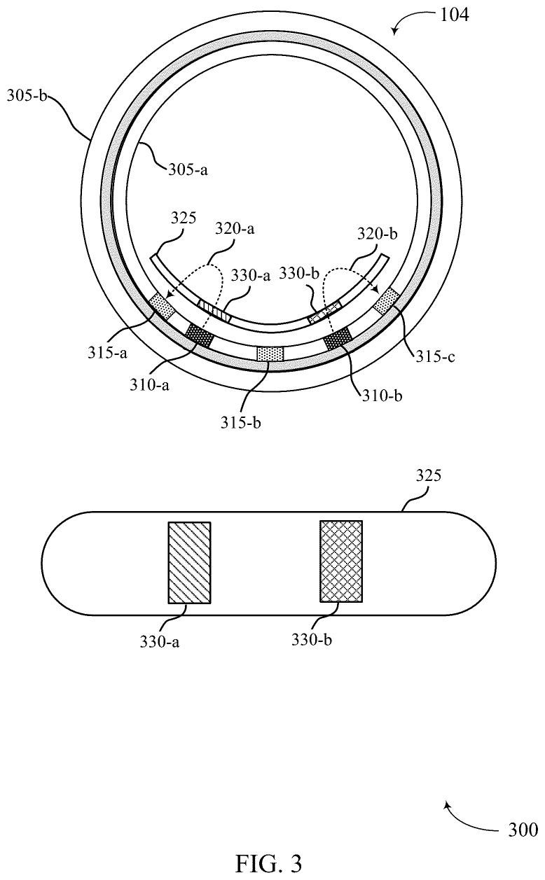

FIG. 3 illustrates an example of a system 300 that supports fluid analysis using a wearable device in accordance with aspects of the present disclosure. The system 300 may implement, or be implemented by, aspects of a system 100 or a system 200. For example, the system 300 may include a wearable device 104 which may be an example of a wearable device 104 or a ring 104 as described with reference to FIGS. 1 and 2, respectively. Further, the wearable device 104 may include light-emitting components 310 and photodetectors 315 which may be examples of optical transmitters and optical receivers included in a PPG system 235 as described with reference to FIG. 2.

In some examples, a user of the wearable device 104 may sweat from one or more sweat glands located on the user's body. Sweat glands may be located on the upper arms, forehead, cheeks, chest, forearm, upper leg, lower back, palm, etc. of the user with the highest concentration of sweat glands being located near or around the user's palm. In some examples, sweat may be made up of several different substances. For example, sweat may include minerals such as sodium, chloride, potassium, ammonium, or calcium. Further, sweat may include compounds such as salt, urea, ethanol (e.g., alcohol), lactate, glucose, or hormones (e.g., cortisol). The substances within the user's sweat may be indicative of the user's health. For example, if the user's salt (e.g., sodium chloride) intake is high (e.g., above a threshold), the user may be at risk of poor cardiovascular health. Consuming foods high in salt may lead to increased blood pressure (e.g., hypertension), blood vessel damage, alteration of body fat metabolism, and gastric mucosal damage potentially resulting in cardiac damage, brain damage, kidney damage, stomach cancer, osteoporosis, or obesity. On the contrary, if the user's salt intake is low, the user may be at risk of nausea, headaches, loss of energy, muscle weakness, irritability, and cramps. Thus, it may be beneficial for a wearable device 104 to track the presence and/or composition of substances within the user's sweat (or other bodily fluids of the user) such that the user may make informed health or lifestyle changes (e.g., increase or decrease their amount of salt intake).

As described herein, the wearable device 104 may determine a presence or concentration of one or more components (or substances) within a bodily fluid of the user (e.g., sweat, saliva, or blood). As shown in FIG. 3, the wearable device 104 may include a light-emitting component 310-a and a light-emitting component 310-b. The light-emitting components 310 may be configured to emit (or transmit) light through a portion of the user's finger. In some examples, the light-emitting components 310 may include one or more LEDs such that the light-emitting components 310 may transmit light of different wavelengths. As one example, the light-emitting components 310 may include one or more of a red diode, a green diode, or an IR diode for transmitting red light, green light, or IR light, respectively.

Additionally, the wearable device 104 may include a photodetector 315-a, a photodetector 315-b, and a photodetector 315-c. The photodetectors 315 may be configured to receive light from one or more of the light-emitting components 310. The photodetectors 315 may also be configured to generate a signal in response to the light received from a light-emitting component 310. The signal may include an indication of a signal strength of the light (e.g., a signal intensity of the light) received from the light-emitting component 310. The photodetectors 315 and the light-emitting components 310 may be embedded in the inner-housing 305-a of the wearable device 104. Further, the photodetectors 315 and the light-emitting components 310 may be located on a bottom-side of the wearable device 104 such that the light-emitting component 310 may transmit light on the palm-side of the user's finger. As shown in FIG. 3, the light-emitting component 310-a may be located between the photodetector 315-a and the photodetector 315-b and the light-emitting component 310-b may be located between the photodetector 315-b and the photodetector 315-c. Such placement of the light-emitting components 310 and the photodetector 315 may allow for a signal path 320-a between the light-emitting component 310-a and the photodetector 315-a and a signal path 320-b between the light-emitting component 310-b and the photodetector 315-c.

The system 300 may also include a fluid collection component 325. The fluid collection component 325 may be used to collect the bodily fluid from the user. In some examples, the material of the fluid collection component 325 may be flexible. For example, the fluid collection component 325 may be made out of soft materials such as woven fabric or latex rubber. In such cases, the flexible fluid collection component 325 may additionally include a layer of adhesive material (e.g., an acrylic polymer) on a surface of the flexible fluid collection component. In another example, the material of the fluid collection component 325 may be rigid. For example, the fluid collection component 325 may include a hard material such as metal or plastic.

In some examples, the fluid collection component 325 may be curved. For example, the rigid fluid collection component 325 may molded into a curved shape similar to the curved shape of the inner-housing 305-a of the wearable device 104 such that that the fluid collection component 325 may be fixed (or coupled) to the inner-housing 305-a of the wearable device 104. Alternatively, the flexible fluid collection component 325 may be wrapped around the user's fingers and secured to the user's finger using the adhesive material.

In some examples, the fluid collection component 325 may include one or more measurement channels 330. For example, as shown in FIG. 3, the fluid collection component may include a measurement channel 330-a and a measurement channel 330-b. In some examples, the measurement channels 330 may include an absorbent material such as cotton or rayon. Further. the measurement channel 330 may be coated or embedded with a chemical indicator that changes the color of the measurement channel 330 depending on a presence or concentration of a substance (e.g., salt or cortisol) of the bodily fluid (e.g., sweat) collected by the fluid collection component 325. For example, prior to exposure, the measurement channel 330 may be white and when exposed to a substance (e.g., sodium, alcohol, etc.), the measurement channel 330 may change to a different color (e.g., red, green, yellow, or purple). Further, the tone of the color (e.g., how light or how dark the color is) may be indicative of the concentration of the substance. For example, a light tone (e.g., pink or light red color) may indicate a low concentration of the substance and a dark tone (e.g., dark red color) may indicate a high concentration of the substance.

In some examples, when exposed to substances, the measurement channel 330 may exhibit changes that are not visible to the human eye. For example, the measurement channel 330 may change “colors” that are not perceptible to the human eye, but may be detected at an IR or UV level. Such changes may be detected using light associated with IR and/or UV wavelengths (e.g., absorption or signal strength of IR/UV wavelengths), similar to UV markings on U.S. paper currency that are only visible when the currency is exposed to UV light.

The wearable device 104 (or a user device associated with the wearable device 104) may instruct the user of the wearable device 104 to place the fluid collection component 325 on a finger of the user. This may include placing a combination of the fluid collection component 325 and the wearable device 104 on the user's finger (e.g., if the fluid collection component 325 is rigid) or wrapping the fluid collection component 325 around the user's finger (e.g., if the fluid collection component 325 is flexible). The fluid collection component 325 may be placed such that the measurement channels 330 are in contact with the user's finger and such that the measurement channels 330 are located on the palm-side of the user's finger.

Once the fluid collection component 325 is placed on the user's finger, the wearable device 104 (or a user device associated with the wearable device 104) may instruct the user to perform a type of physical activity for a duration (e.g., 30 minutes). In some examples, the physical activity may cause the user to sweat and the measurement channels 330 may collect the sweat during the duration of the physical activity. In some examples, the wearable device 104 may be on a different finger than the finger in contact with the fluid collection component 325 during the duration of the physical activity. In such examples, the wearable device 104 may acquire physiological data (e.g., PPG data, heart rate data, HRV data, blood oxygen concentration data) from the user during the duration. That is, the wearable device 104 may not stop or delay the gathering of physiological data from the user during the duration of the physical activity.

Following the physical activity, the wearable device 104 may prompt the user to place the wearable device 104 on the finger with the fluid collection component 325 (e.g., if the fluid collection component 325 and the wearable device 104 are separate from one another). In response to the prompt, the user may slip the wearable device 104 over the top of the fluid collection component 325. Once the wearable device 104 is positioned on top of the fluid collection component 325, the wearable device 104 may prompt the user to rotate the wearable device 104. The user may rotate the wearable device 104 until at least one measurement channel 330 is proximate to (or between) at least one light-emitting component 310 and at least one photodetector 315. For example, as shown in FIG. 3, the wearable device 104 may be rotated such that the measurement channel 330-a is proximate to the light-emitting component 310-a and the photodetector 315-a. Additionally or alternatively, the wearable device 104 may be rotated such that the measurement channel 330-b is proximate to the light-emitting component 310-b and the photodetector 315-c.

Once in position, the wearable device 104 may transmit, via the light-emitting component 310-a, a light (e.g., a red light, a green light, or an IR light) and receive, via the photodetector 315-a, the light from the light-emitting component 310-a. The light may travel along the signal path 320-a from the light-emitting component 310-a to the photodetector 315-a. The signal path 320-a may pass through the measurement channel 330-a. Additionally, or alternatively, the signal path 320-a may be formed by light transmitted by the light-emitting component 310-a and reflecting or refracting off the measurement channel 330-a to the photodetector 315-a. Moreover, in some cases, the signal path 320-a between the light-emitting component 310-a and the photodetector may include a combination of both transmissive and reflective light (e.g., may include light that is transmitted through the measurement channel 330-a, and light that is reflected off the measurement channel 330-a).

Upon receiving the light, the photodetector 315-a (or a processing module in communication with the photodetector) may analyze characteristics of the received light and generate a signal indicating the characteristics. In some examples, the wearable device 104 may determine the presence of a substance included in the user's bodily fluid based on signals generated by the photodetectors 315. In particular, the presence and/or concentration of substances in the user's bodily fluid may cause the color of the measurement channels 330 (or other parameters of the measurement channels 330 not visible to the human eye) to change, which affects the amount of light reflected/absorbed by the measurement channels, thereby affecting the light received at the photodetectors 315.

For example, the presence or concentration of a substance in the user's bodily fluid may turn a color of the measurement channel 330-a from a first color (e.g., white) to a second color (e.g., red, green, yellow, or purple). As an example, the presence of a substance in the user's bodily fluid may turn the color of the measurement channel 330-a green. A green measurement channel 330-a may reflect green light and absorb other colors of light (e.g., red light or IR light). Thus, the wearable device 104 may transmit, via the light-emitting component, at least a green light along the signal path 320-a. The photodetector 315-a may receive the green light and determine a light intensity of the received green light (e.g., a signal strength) and generate a signal indicating characteristics of the received green light. If a difference between the light intensity of the received green light and the light intensity of the transmitted green light is below a threshold, the wearable device 104 may determine the presence of the substance. In some examples, a user device associated with the wearable device 104 may display a message indicating the presence of the component.

Additionally or alternatively, the wearable device 104 may determine a concentration of the substance included in the user's bodily fluid. Depending on the concentration of the substance, the measurement channel 330 may turn a different shade of a color or colors. Each shade may absorb colored light differently. For example, a first shade may absorb a first amount of green light and a second amount of red light and a second shade may absorb a third amount of green light and a fourth amount of red light. Thus, the wearable device 104 may transmit, via the light-emitting component 310-a, a series of light along the signal path 320. In some examples, the wearable device 104 may transmit light associated with two or more wavelengths. For example, the wearable device 104 may transmit a red light, followed by a green light, followed by an IR light. In such cases, the photodetectors 315 may be configured to determine relative quantities or proportions of the light of the different wavelengths that are reflected/absorbed by the measurement channels 330 (e.g., red absorption vs. IR absorption), and may use the relative reflection/absorption metrics to determine a color of the measurement channels 330 (and therefore the presence/concentration of substances)

The photodetector 315-a may receive the light and determine a light intensity (e.g., signal strength) of each of the received red light, green light, and IR light and generate a signal for each light indicating a characteristic of the respective light (e.g., light intensity). Further, the wearable device 104 (or a processing module of the wearable device 104) may determine differences between the light intensity of the received red light, green light, and IR light and the transmitted red light, green light, and IR light, respectively (e.g., the reflectance of the red light, green light, and IR light). The wearable device 104 (or the processing module) may compare the differences to one another and determine a shade (e.g., a color) of the measurement channel 330-a based on the comparison. The shade of the measurement channel 330-a may be indicative of the concentration of the component. In some examples, a user device associated with the wearable device 104 may display a message indicating the concentration of the component.

In some examples, the color of the measurement channel 330-a may change due to factors other than the presence or concentration of the substance of the bodily fluid. For example, as the measurement channel 330 ages, the color of the measurement channel 330 may change from a white color to a yellow color. As another example, dirt or other debris may get on the measurement channel 330-a causing the color of the measurement channel 330 to change. Such factors are not associated with the presence or concentration of the component and may negatively impact the ability of the wearable device 104 to detect the concentration or presence of the substance.

As such, to account for other color changing variables that are not attributable to substances within the user's bodily fluid (e.g., dirt, oil, etc.) the wearable device 104 may include a reference measurement channel 330 (e.g., the measurement channel 330-b). The measurement channel 330-b may include the same material as the measurement channel 330-b. In some cases, a reference measurement channel 330 may not be configured to change colors when exposed to substances within the use's bodily fluid. That is, the reference measurement channel 330 may be configured to remain the same color (or approximately the same color) when exposed to substances within the bodily fluid, whereas the measurement channel 330-a may be configured to change colors when exposed to the substances. However, the reference measurement channel 330 may change color when exposed to dirt, oil, and other debris, and may therefore provide a reference that indicates how much of the color change in the measurement channel 330-a is attributable to the substances within the bodily fluid, and how much of the color change is attributable to other variables.

For example, the wearable device 104 may transmit, via the light-emitting component 310-b, light associated with one or more wavelengths along the signal path 320-b. The photodetector 315-c may receive the light and determine a light intensity for each of the one or more wavelengths of light (e.g., signal strength) and determine differences between the light intensity of the received one or more wavelengths of light and the light intensity of the transmitted one or more wavelengths light. When determining the presence or concentration of the substance of the bodily fluid collected by the measurement channel 330-a, the wearable device 104 may take into account the signal generated using the reference measurement channel 330-b. In other words, signals generated using light transmitted through (or reflected from) the reference measurement channel 330-b may be used to calibrate or adjust signals generated using the measurement channel 330-a.

In additional or alternative implementations, the wearable device 104 may utilize the measurement channel 330-b to detect a presence or concentration of a substance that is different or the same as the component detected by the measurement channel 330-a. In other words, the fluid collection component 325 may include multiple measurement channels 330 configured to measure the presence/concentration of multiple substances. Moreover, in some cases, the fluid collection component 325 may include a single reference measurement channel 330 that is used to calibrate signals for multiple measurement channels 330-a, 330-b that are used to measure one or more substances.

In some examples, the fluid collection component 325 may include micro-fluidic channels (not shown in FIG. 3). Material (e.g., resin or silicon) may be deposited on the surface of the fluid collection component 325 to create the microfluidic channels and the microfluidic channels may collect and direct the bodily fluid of the user to one or both of the measurement channels 330. In some examples, the microfluidic channels may cover a larger portion of the fluid collection component 325 than the measurement channels 330 which may increase the amount of bodily fluid collected by the measurement channels 330.

In another example, the microfluidic channels may collect the sweat of the user (e.g., during physical exercise) and the wearable device 104 may run electricity through the microfluidic channels using conductive patches (e.g., electrodes). In other words, the wearable device 104 may utilize electrodes to conduct electricity through the bodily fluid collected by the fluid collection component 325, where levels of electrical conductance (e.g., impedance, electrical resistance) may be used to determine the presence/concentration of substances within the bodily fluid.

For example, the wearable device 104 may include one or more electrodes (e.g., conductive patches) disposed within the inner-housing 305-a of the wearable device 104, where the electrodes are coupled to internal circuitry of the wearable device 104 (e.g., a battery of the wearable device). A first electrode may come in contact with a first portion of a microfluidic channel (and/or measurement channel 330-a) and the second electrode may come in contact with a second portion of the microfluidic channel/measurement channel 330-a. The wearable device 104 may apply a voltage to the first electrode to generate an electrical current from the first electrode through the microfluidic channel/measurement channel 330 to the second electrode. Different concentrations of a component included in the user bodily fluid may modulate the conductance of electricity. After applying the voltage, the wearable device 104 may measure a conductance value of the electrical current (e.g., voltage of electrical current received at the second electrode) and determine the concentration of the substance included in the bodily fluid of the user based on the conductance value.

In some examples, the wearable device 104 may perform iontophoresis, where an electrical current/voltage is used to stimulate sweat glands to produce sweat that may be collected by the fluid collection component 325. In such examples, one or more of the electrodes/conductive patches may be exposed to the user's finger. The wearable device 104 may apply a voltage to the one or more electrodes to draw out the bodily fluid from the user (e.g., stimulate one or more sweat glands of the user). In some examples, the wearable device 104 may apply the current while the user is performing the physical activity.

While much of the present disclosure is described in the context of sweat analysis, this is not to be regarded as a limitation of the present disclosure, unless noted otherwise herein. In particular, fluid analysis techniques described herein may be used to analyze substances within other types of bodily fluids, including saliva, blood, urine, and the like. For example, a user may be instructed to lick the fluid analysis component 325 and place the fluid analysis component 325 proximate to the wearable device 104 so that the wearable device 104 may be used to determine the presence and/or concentration of substances within the user's saliva. Moreover, the fluid analysis techniques described herein may be implemented in the context of other types of wearable devices 104, such as wearable watch devices, chest straps, arm bands, leg bands, necklaces, and the like.

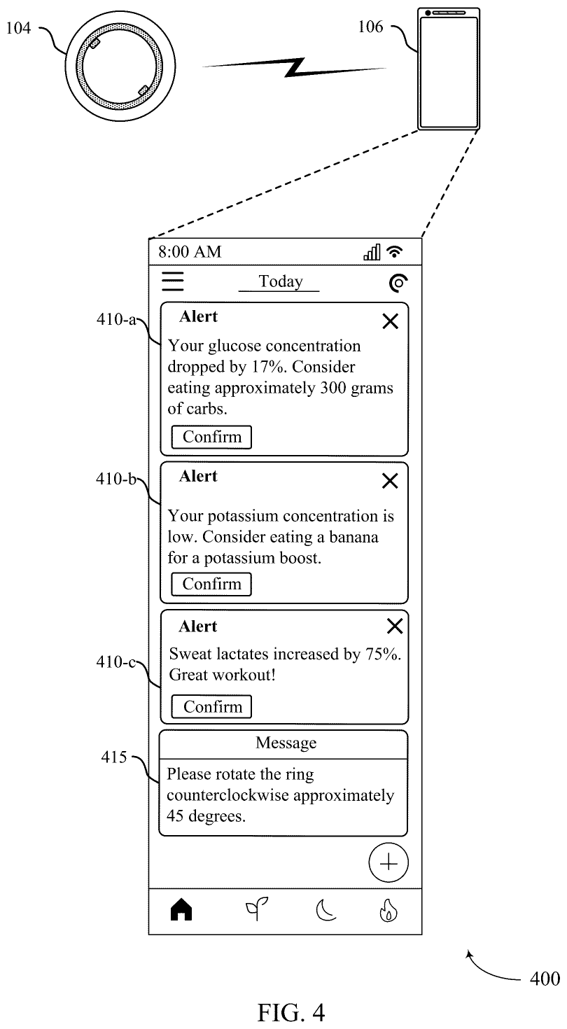

FIG. 4 illustrates an example of a GUI 400 that supports fluid analysis using a wearable device in accordance with aspects of the present disclosure. The GUI 400 may implement, or be implemented by, aspects of the system 100, the system 200, and the system 300. For example, the GUI 400 may be an example of a GUI 275 of a user device 106 corresponding to a user 102 as described with reference to FIG. 2. Further, FIG. 4 may include a wearable device 104 which may be an example of a wearable device 104 or a ring 104 as described with reference to FIGS. 1 through 3. Moreover, FIG. 4 may include a user device 106 which may be an example of a user device 106 as described with reference to FIGS. 1 and 2. In some examples, the GUI 400 may illustrate a series of application pages 405 which may be displayed to a user via GUI 400.

As described with reference to FIG. 2, a user may be associated with the wearable device 104 and a user device 106. In some examples, the wearable device 104 and the user device 106 may establish a communication link and communicate with one another using the communication link. In some examples, the wearable device 104 may send raw and/or processed data to the user device 106. For example, the wearable device 104 may transmit bodily fluid content data to the user device 106. An example of the bodily fluid content data may be a concentration or a presence of a substance (e.g., sodium chloride, cortisol, lactate, sodium, potassium, calcium, urea, ethanol/alcohol, ammonium, glucose, inflammatory markers, or toxic substance) included in a user's bodily fluid. Additionally, or alternatively, the wearable device 104 may transmit indications of the signals generated by the photodetectors of the wearable device (e.g., bodily fluid content data includes generated signals) so that the user device may analyze the signals to determine the presence/concentration of substances. The wearable device may obtain the bodily fluid content data from the user using the methods as described in FIG. 3.

Upon receiving the bodily fluid content data (e.g., signals generated by the photodetectors of the wearable device 104), the user device 106 may generate one or more alerts 410 or one or more messages 415 for display on the GUI 400. To access the messages 415 and the alerts 410, the user may open an application (e.g., wearable application 250) associated with the wearable device 104. An application page 405 (e.g., an application homepage) of the application may display the one or more messages 415 and the one or more alerts 410 associated with the bodily fluid content data.

In some examples, the bodily fluid content data may indicate that a glucose concentration of the user decreased by 17%. In such examples, the application page 405 may display an alert 410-a. The alert 410-a may include text that reads “Your glucose concentration dropped by 17%.” Further, the alert 410-a may include a recommendation to the user. For example, the alert 410-a may include text that reads “Consider eating approximately 300 grams of carbs.” In another example, the bodily fluid content data may indicate that potassium concentration of the user is below a threshold. In such examples, the application page 405 may display an alert 410-b. The alert 410-b may include text that reads “Your potassium concentration is low.” Further, the alert 410-b may include a recommendation to the user. For example, the alert 410-b may include text that reads “Consider eating a banana for a potassium boost.” In another example, the bodily fluid content may indicate that a lactate concentration of the user is at a preferred level (e.g., below a first threshold and below a second threshold). In such examples, the application page 405 may display an alert 410-c. The alert 410-c may include texts that read “Sweat lactates increased by 75%. Great workout!”

As described with reference to FIG. 3, the user may place a fluid collection component (e.g., an adhesive patch or a microfluidic chamber) on the user's finger and collect bodily fluid from the user for a period of time. When an adequate amount of bodily fluid is collected from the user, the user may place the wearable device 104 on the user's finger, over the top of the fluid collection component. In some examples, the orientation of the wearable device 104 when placed on the user's finger may not be the most optimal for measurement. For example, a measurement channel of the fluid collection component may not be proximal to one or both of a light-emitting component or a photodetector. In such examples, the application page 405 may display a message 415 instructing the user to reorientate the wearable device. For example, the message 415 may include text that reads “Please rotate the ring counterclockwise approximately 45 degrees.”

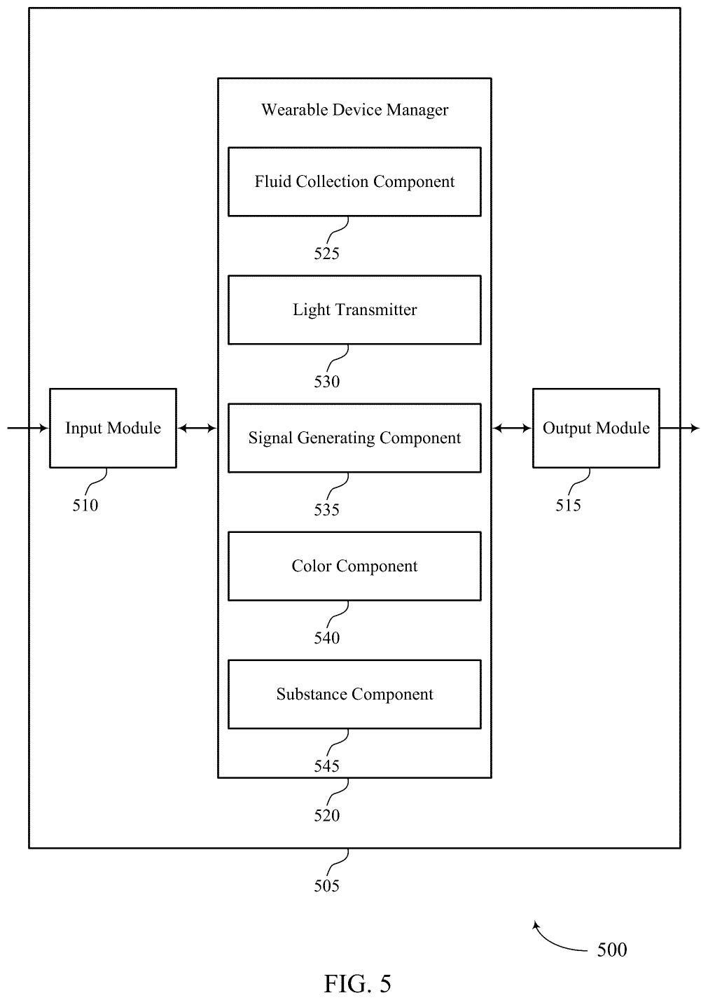

FIG. 5 illustrates a block diagram 500 of a device 505 that supports fluid analysis using a wearable device in accordance with aspects of the present disclosure. The device 505 may include an input module 510, an output module 515, and a wearable device manager 520. The device 505 may also include a processor. Each of these components may be in communication with one another (e.g., via one or more buses).

For example, the wearable device manager 520 may include a fluid collection component 525, a light transmitter 530, a signal generating component 535, a color component 540, a substance component 545, or any combination thereof. In some examples, the wearable device manager 520, or various components thereof, may be configured to perform various operations (e.g., receiving, monitoring, transmitting) using or otherwise in cooperation with the input module 510, the output module 515, or both. For example, the wearable device manager 520 may receive information from the input module 510, send information to the output module 515, or be integrated in combination with the input module 510, the output module 515, or both to receive information, transmit information, or perform various other operations as described herein.

The wearable device manager 520 may support fluid analysis in accordance with examples as disclosed herein. The fluid collection component 525 may be configured as or otherwise support a means for collecting a bodily fluid of a user using a fluid collection component, wherein the fluid collection component comprises one or more measurement channels configured to change color when exposed to the bodily fluid. The light transmitter 530 may be configured as or otherwise support a means for transmitting light associated with one or more wavelengths using one or more light-emitting components of a wearable device. The signal generating component 535 may be configured as or otherwise support a means for generating, using a photodetector of the wearable device, one or more signals based at least in part on the light reflected off the one or more measurement channels of the fluid collection component and received by the photodetector. The color component 540 may be configured as or otherwise support a means for determining a color of the one or more measurement channels based at least in part on the one or more signals. The substance component 545 may be configured as or otherwise support a means for determining a presence or concentration of one or more substances within the bodily fluid based at least in part on the color.

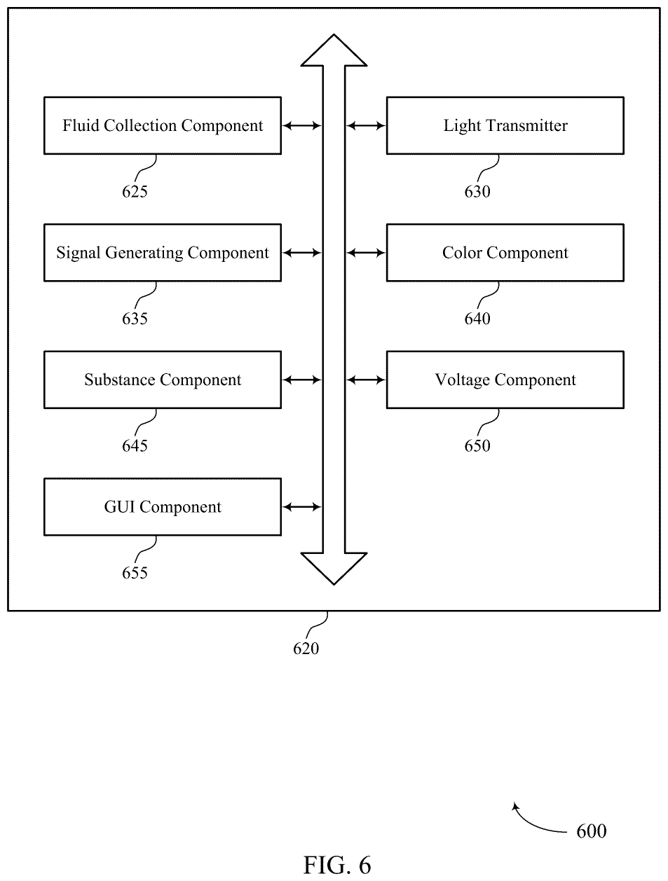

FIG. 6 illustrates a block diagram 600 of a wearable device manager 620 that supports fluid analysis using a wearable device in accordance with aspects of the present disclosure. The wearable device manager 620 may be an example of aspects of a wearable device manager or a wearable device manager 520, or both, as described herein. The wearable device manager 620, or various components thereof, may be an example of means for performing various aspects of fluid analysis using a wearable device as described herein. For example, the wearable device manager 620 may include a fluid collection component 625, a light transmitter 630, a signal generating component 635, a color component 640, a substance component 645, a voltage component 650, a GUI component 655, or any combination thereof. Each of these components may communicate, directly or indirectly, with one another (e.g., via one or more buses).

The wearable device manager 620 may support fluid analysis in accordance with examples as disclosed herein. The fluid collection component 625 may be configured as or otherwise support a means for collecting a bodily fluid of a user using a fluid collection component, wherein the fluid collection component comprises one or more measurement channels configured to change color when exposed to the bodily fluid. The light transmitter 630 may be configured as or otherwise support a means for transmitting light associated with one or more wavelengths using one or more light-emitting components of a wearable device. The signal generating component 635 may be configured as or otherwise support a means for generating, using a photodetector of the wearable device, one or more signals based at least in part on the light reflected off the one or more measurement channels of the fluid collection component and received by the photodetector. The color component 640 may be configured as or otherwise support a means for determining a color of the one or more measurement channels based at least in part on the one or more signals. The substance component 645 may be configured as or otherwise support a means for determining a presence or concentration of one or more substances within the bodily fluid based at least in part on the color.

In some examples, the light transmitter 630 may be configured as or otherwise support a means for transmitting first light associated with a first wavelength. In some examples, the signal generating component 635 may be configured as or otherwise support a means for generating a first signal based at least in part on the first light reflected off the one or more measurement channels and received by the photodetector. In some examples, the light transmitter 630 may be configured as or otherwise support a means for transmitting second light associated with a second wavelength. In some examples, the signal generating component 635 may be configured as or otherwise support a means for generating a second signal based at least in part on the second light reflected off the one or more measurement channels and received by the photodetector, wherein determining the color is based at least in part on a comparison of the first signal and the second signal. In some examples, the first wavelength or the second wavelength comprise a wavelength associated with IR light.

In some examples, the one or more measurement channels comprise a reference channel that is configured to not change color when exposed to the bodily fluid, and the signal generating component 635 may be configured as or otherwise support a means for generating one or more additional signals based at least in part on the light reflected off the reference channel and received by the photodetector, wherein determining the color of the one or more measurement channels is based at least in part on a comparison of the one or more signals and the one or more additional signals.

In some examples, the voltage component 650 may be configured as or otherwise support a means for generating an electrical current using a first electrode of the wearable device. In some examples, the signal generating component 635 may be configured as or otherwise support a means for generating a second signal based at least in part on the electrical current received through the one or more measurement channels using a second electrode of the wearable device, wherein determining the presence or the concentration of the one or more substances is based at least in part on the second signal.

In some examples, the voltage component 650 may be configured as or otherwise support a means for generating an electrical current using an electrode of the wearable device, wherein the electrical current is configured to stimulate one or more sweat glands of the user, wherein collecting the bodily fluid is based at least in part on generating the electrical current.

In some examples, the GUI component 655 may be configured as or otherwise support a means for causing a user device associated with the wearable device to display a message associated with the presence or the concentration of the one or more substances.

In some examples, the GUI component 655 may be configured as or otherwise support a means for causing a user device associated with the wearable device to display instructions for the user to manipulate the fluid collection component to collect the bodily fluid, to manipulate the wearable device relative to the one or more measurement channels, or both, wherein collecting the bodily fluid, transmitting the light, generating the one or more signals, or any combination thereof, is based at least in part on the instructions.

In some examples, the one or more light-emitting components and the photodetector are configured to acquire physiological data from the user, the physiological data comprising PPG data, heart rate data, HRV, blood oxygen saturation data, or any combination thereof.

In some examples, the fluid collection component comprises an adhesive patch, a lateral flow patch, a microfluidic chamber, a material disposed on a surface of the wearable device, or any combination thereof.

In some examples, the fluid collection component comprises an attachment component configured to couple with the wearable device. In some examples, the attachment component is configured to position the one or more measurement channels proximate to the one or more light-emitting components, the photodetector, or both. In some examples, the bodily fluid comprises perspiration, saliva, tears, blood, or any combination thereof.

In some examples, the one or more substances comprise sodium chloride, cortisol, lactate, sodium, potassium, calcium, urea, alcohol, ammonium, glucose, inflammatory markers, toxic substances, or any combination thereof. In some examples, the wearable device comprises a wearable ring device.

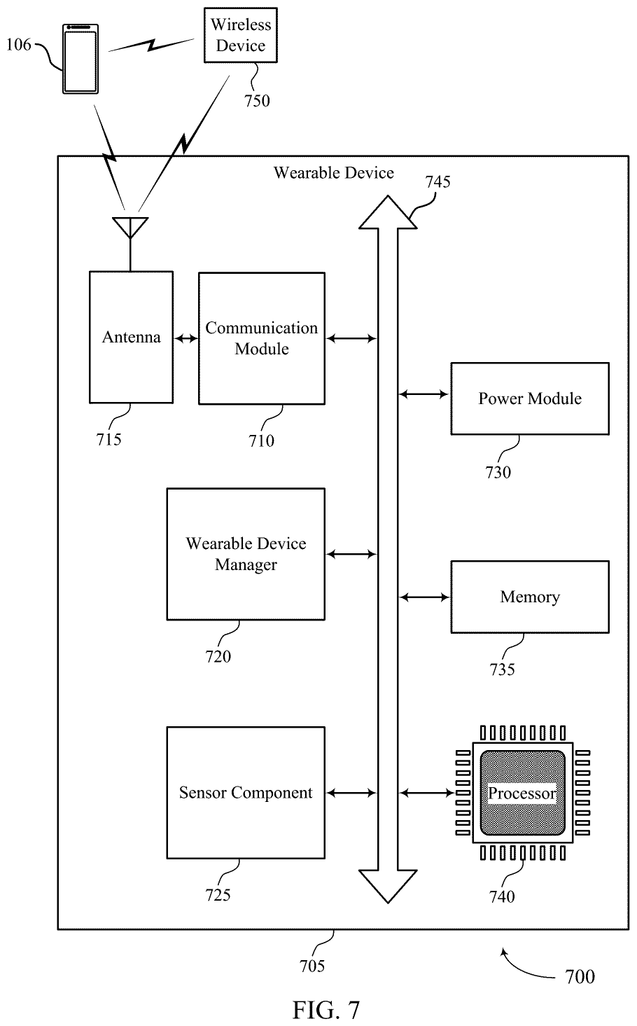

FIG. 7 illustrates a diagram of a system 700 including a device 705 that supports fluid analysis using a wearable device in accordance with aspects of the present disclosure. The device 705 may be an example of or include the components of a device 505 as described herein. The device 705 may include an example of a wearable device 104, as described previously herein. The device 705 may include components for bi-directional communications including components for transmitting and receiving communications with a user device 106 and a server 110, such as a wearable device manager 720, a communication module 710, an antenna 715, a sensor component 725, a power module 730, a memory 735, a processor 740, and a wireless device 750. These components may be in electronic communication or otherwise coupled (e.g., operatively, communicatively, functionally, electronically, electrically) via one or more buses (e.g., a bus 745).

The wearable device manager 720 may support fluid analysis in accordance with examples as disclosed herein. For example, the wearable device manager 720 may be configured as or otherwise support a means for collecting a bodily fluid of a user using a fluid collection component, wherein the fluid collection component comprises one or more measurement channels configured to change color when exposed to the bodily fluid. The wearable device manager 720 may be configured as or otherwise support a means for transmitting light associated with one or more wavelengths using one or more light-emitting components of a wearable device. The wearable device manager 720 may be configured as or otherwise support a means for generating, using a photodetector of the wearable device, one or more signals based at least in part on the light reflected off the one or more measurement channels of the fluid collection component and received by the photodetector. The wearable device manager 720 may be configured as or otherwise support a means for determining a color of the one or more measurement channels based at least in part on the one or more signals. The wearable device manager 720 may be configured as or otherwise support a means for determining a presence or concentration of one or more substances within the bodily fluid based at least in part on the color.

By including or configuring the wearable device manager 720 in accordance with examples as described herein, the device 705 may support techniques for determining a presence or concentration of a component included in a user's bodily fluid and displaying a message associated with the presence or concentration of the component to the user such that the user may make informed decisions on their health and potentially avoid physiological problems.

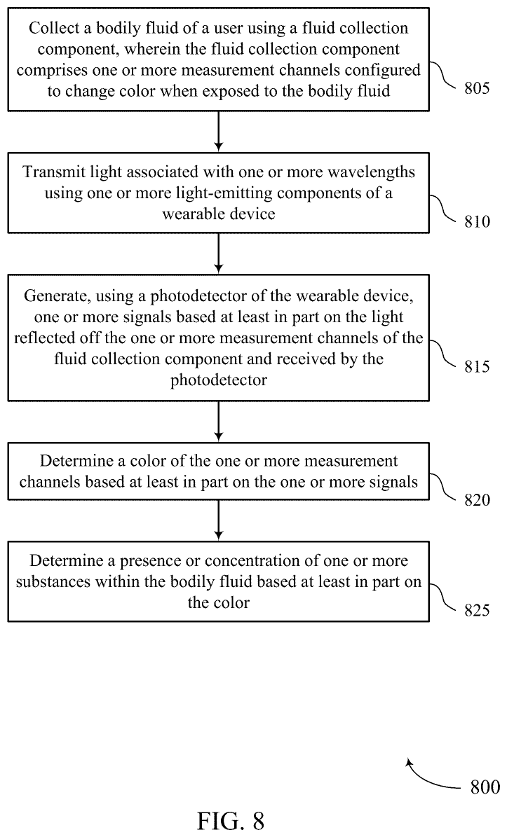

FIG. 8 illustrates a flowchart showing a method 800 that supports fluid analysis using a wearable device in accordance with aspects of the present disclosure. The operations of the method 800 may be implemented by a wearable device or its components as described herein. For example, the operations of the method 800 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 805, the method may include collecting a bodily fluid of a user using a fluid collection component, wherein the fluid collection component comprises one or more measurement channels configured to change color when exposed to the bodily fluid. The operations of 805 may be performed in accordance with examples as disclosed herein. In some examples, aspects of the operations of 805 may be performed by a fluid collection component 625 as described with reference to FIG. 6.

At 810, the method may include transmitting light associated with one or more wavelengths using one or more light-emitting components of a wearable device. The operations of 810 may be performed in accordance with examples as disclosed herein. In some examples, aspects of the operations of 810 may be performed by a light transmitter 630 as described with reference to FIG. 6.

At 815, the method may include generating, using a photodetector of the wearable device, one or more signals based at least in part on the light reflected off the one or more measurement channels of the fluid collection component and received by the photodetector. The operations of 815 may be performed in accordance with examples as disclosed herein. In some examples, aspects of the operations of 815 may be performed by a signal generating component 635 as described with reference to FIG. 6.

At 820, the method may include determining a color of the one or more measurement channels based at least in part on the one or more signals. The operations of 820 may be performed in accordance with examples as disclosed herein. In some examples, aspects of the operations of 820 may be performed by a color component 640 as described with reference to FIG. 6.

At 825, the method may include determining a presence or concentration of one or more substances within the bodily fluid based at least in part on the color. The operations of 825 may be performed in accordance with examples as disclosed herein. In some examples, aspects of the operations of 825 may be performed by a substance component 645 as described with reference to FIG. 6.

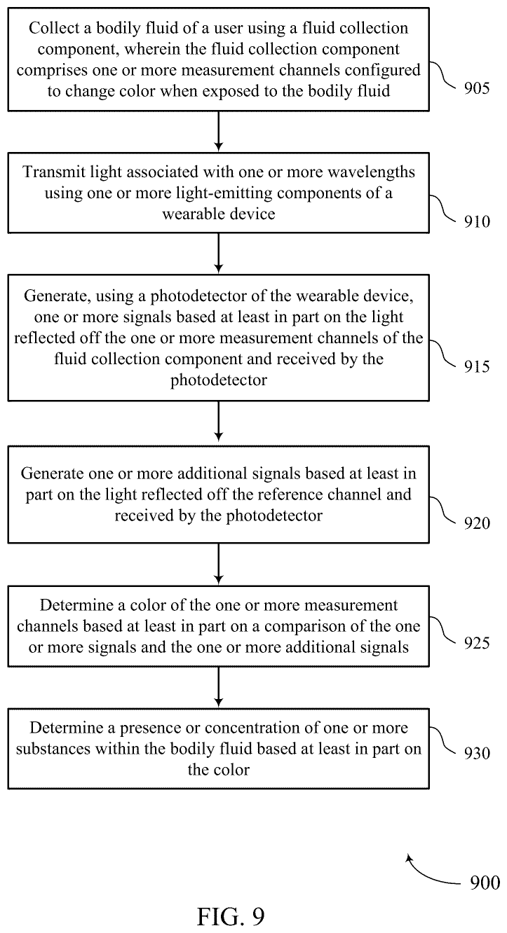

FIG. 9 illustrates a flowchart showing a method 900 that supports fluid analysis using a wearable device in accordance with aspects of the present disclosure. The operations of the method 900 may be implemented by a wearable device or its components as described herein. For example, the operations of the method 900 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 905, the method may include collecting a bodily fluid of a user using a fluid collection component, wherein the fluid collection component comprises one or more measurement channels configured to change color when exposed to the bodily fluid. The operations of 905 may be performed in accordance with examples as disclosed herein. In some examples, aspects of the operations of 905 may be performed by a fluid collection component 625 as described with reference to FIG. 6.

At 910, the method may include transmitting light associated with one or more wavelengths using one or more light-emitting components of a wearable device. The operations of 910 may be performed in accordance with examples as disclosed herein. In some examples, aspects of the operations of 910 may be performed by a light transmitter 630 as described with reference to FIG. 6.

At 915, the method may include generating, using a photodetector of the wearable device, one or more signals based at least in part on the light reflected off the one or more measurement channels of the fluid collection component and received by the photodetector. The operations of 915 may be performed in accordance with examples as disclosed herein. In some examples, aspects of the operations of 915 may be performed by a signal generating component 635 as described with reference to FIG. 6.

At 920, the method may include generating one or more additional signals based at least in part on the light reflected off a reference channel and received by the photodetector. The operations of 920 may be performed in accordance with examples as disclosed herein. In some examples, aspects of the operations of 920 may be performed by a signal generating component 635 as described with reference to FIG. 6.

At 925, the method may include determining a color of the one or more measurement channels based at least in part on a comparison of the one or more signals and the one or more additional signals. The operations of 925 may be performed in accordance with examples as disclosed herein. In some examples, aspects of the operations of 925 may be performed by a color component 640 as described with reference to FIG. 6.

At 930, the method may include determining a presence or concentration of one or more substances within the bodily fluid based at least in part on the color. The operations of 930 may be performed in accordance with examples as disclosed herein. In some examples, aspects of the operations of 930 may be performed by a substance component 645 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for fluid analysis is described. The method may include collecting a bodily fluid of a user using a fluid collection component, wherein the fluid collection component comprises one or more measurement channels configured to change color when exposed to the bodily fluid, transmitting light associated with one or more wavelengths using one or more light-emitting components of a wearable device, generating, using a photodetector of the wearable device, one or more signals based at least in part on the light reflected off the one or more measurement channels of the fluid collection component and received by the photodetector, determining a color of the one or more measurement channels based at least in part on the one or more signals, and determining a presence or concentration of one or more substances within the bodily fluid based at least in part on the color.

An apparatus for fluid analysis is described. The apparatus may include a processor, memory coupled with the processor, and instructions stored in the memory. The instructions may be executable by the processor to cause the apparatus to collect a bodily fluid of a user using a fluid collection component, wherein the fluid collection component comprises one or more measurement channels configured to change color when exposed to the bodily fluid, transmit light associated with one or more wavelengths using one or more light-emitting components of a wearable device, generate, using a photodetector of the wearable device, one or more signals based at least in part on the light reflected off the one or more measurement channels of the fluid collection component and received by the photodetector, determine a color of the one or more measurement channels based at least in part on the one or more signals, and determine a presence or concentration of one or more substances within the bodily fluid based at least in part on the color.

Another apparatus for fluid analysis is described. The apparatus may include means for collecting a bodily fluid of a user using a fluid collection component, wherein the fluid collection component comprises one or more measurement channels configured to change color when exposed to the bodily fluid, means for transmitting light associated with one or more wavelengths using one or more light-emitting components of a wearable device, means for generating, using a photodetector of the wearable device, one or more signals based at least in part on the light reflected off the one or more measurement channels of the fluid collection component and received by the photodetector, means for determining a color of the one or more measurement channels based at least in part on the one or more signals, and means for determining a presence or concentration of one or more substances within the bodily fluid based at least in part on the color.

A non-transitory computer-readable medium storing code for fluid analysis is described. The code may include instructions executable by a processor to collect a bodily fluid of a user using a fluid collection component, wherein the fluid collection component comprises one or more measurement channels configured to change color when exposed to the bodily fluid, transmit light associated with one or more wavelengths using one or more light-emitting components of a wearable device, generate, using a photodetector of the wearable device, one or more signals based at least in part on the light reflected off the one or more measurement channels of the fluid collection component and received by the photodetector, determine a color of the one or more measurement channels based at least in part on the one or more signals, and determine a presence or concentration of one or more substances within the bodily fluid based at least in part on the color.

Some examples of the method, apparatuses, and non-transitory computer-readable medium described herein may further include operations, features, means, or instructions for transmitting first light associated with a first wavelength, generating a first signal based at least in part on the first light reflected off the one or more measurement channels and received by the photodetector, transmitting second light associated with a second wavelength, and generating a second signal based at least in part on the second light reflected off the one or more measurement channels and received by the photodetector, wherein determining the color may be based at least in part on a comparison of the first signal and the second signal.

In some examples of the method, apparatuses, and non-transitory computer-readable medium described herein, the first wavelength or the second wavelength comprise a wavelength associated with IR light.

In some examples of the method, apparatuses, and non-transitory computer-readable medium described herein, the one or more measurement channels comprise a reference channel that may be configured to not change color when exposed to the bodily fluid and the method, apparatuses, and non-transitory computer-readable medium may include further operations, features, means, or instructions for generating one or more additional signals based at least in part on the light reflected off the reference channel and received by the photodetector, wherein determining the color of the one or more measurement channels may be based at least in part on a comparison of the one or more signals and the one or more additional signals.

Some examples of the method, apparatuses, and non-transitory computer-readable medium described herein may further include operations, features, means, or instructions for generating an electrical current using a first electrode of the wearable device and generating a second signal based at least in part on the electrical current received through the one or more measurement channels using a second electrode of the wearable device, wherein determining the presence or the concentration of the one or more substances may be based at least in part on the second signal.

Some examples of the method, apparatuses, and non-transitory computer-readable medium described herein may further include operations, features, means, or instructions for generating an electrical current using an electrode of the wearable device, wherein the electrical current may be configured to stimulate one or more sweat glands of the user, wherein collecting the bodily fluid may be based at least in part on generating the electrical current.

Some examples of the method, apparatuses, and non-transitory computer-readable medium described herein may further include operations, features, means, or instructions for causing a user device associated with the wearable device to display a message associated with the presence or the concentration of the one or more substances.

Some examples of the method, apparatuses, and non-transitory computer-readable medium described herein may further include operations, features, means, or instructions for causing a user device associated with the wearable device to display instructions for the user to manipulate the fluid collection component to collect the bodily fluid, to manipulate the wearable device relative to the one or more measurement channels, or both, wherein collecting the bodily fluid, transmitting the light, generating the one or more signals, or any combination thereof, may be based at least in part on the instructions.

In some examples of the method, apparatuses, and non-transitory computer-readable medium described herein, the one or more light-emitting components and the photodetector may be configured to acquire physiological data from the user, the physiological data comprising PPG data, heart rate data, HRV, blood oxygen saturation data, or any combination thereof.

In some examples of the method, apparatuses, and non-transitory computer-readable medium described herein, the fluid collection component comprises an adhesive patch, a lateral flow patch, a microfluidic chamber, a material disposed on a surface of the wearable device, or any combination thereof.

In some examples of the method, apparatuses, and non-transitory computer-readable medium described herein, the fluid collection component comprises an attachment component configured to couple with the wearable device.

In some examples of the method, apparatuses, and non-transitory computer-readable medium described herein, the attachment component may be configured to position the one or more measurement channels proximate to the one or more light-emitting components, the photodetector, or both.

In some examples of the method, apparatuses, and non-transitory computer-readable medium described herein, the bodily fluid comprises perspiration, saliva, tears, blood, or any combination thereof.

In some examples of the method, apparatuses, and non-transitory computer-readable medium described herein, the one or more substances comprise sodium chloride, cortisol, lactate, sodium, potassium, calcium, urea, alcohol, ammonium, glucose, inflammatory markers, toxic substances, or any combination thereof.

In some examples of the method, apparatuses, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as IR, radio, and microwave, then the coaxial cable, fiber optic cable, twisted pair, DSL, or wireless technologies such as IR, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for fluid analysis, comprising:
collecting a bodily fluid of a user using a fluid collection component, wherein the fluid collection component comprises one or more measurement channels configured to change color when exposed to the bodily fluid;
transmitting light associated with one or more wavelengths using one or more light-emitting components of a wearable device;
generating, using a photodetector of the wearable device, one or more signals based at least in part on the light reflected off the one or more measurement channels of the fluid collection component and received by the photodetector;
determining a color of the one or more measurement channels based at least in part on the one or more signals; and
determining a presence or concentration of one or more substances within the bodily fluid based at least in part on the color.

2. The method of claim 1, further comprising:
transmitting first light associated with a first wavelength;
generating a first signal based at least in part on the first light reflected off the one or more measurement channels and received by the photodetector;
transmitting second light associated with a second wavelength; and
generating a second signal based at least in part on the second light reflected off the one or more measurement channels and received by the photodetector, wherein determining the color is based at least in part on a comparison of the first signal and the second signal.

3. The method of claim 2, wherein the first wavelength or the second wavelength comprise a wavelength associated with infrared light.

4. The method of claim 1, wherein the one or more measurement channels comprise a reference channel that is configured to not change color when exposed to the bodily fluid, the method further comprising:
generating one or more additional signals based at least in part on the light reflected off the reference channel and received by the photodetector, wherein determining the color of the one or more measurement channels is based at least in part on a comparison of the one or more signals and the one or more additional signals.

5. The method of claim 1, further comprising:
generating an electrical current using a first electrode of the wearable device; and
generating a second signal based at least in part on the electrical current received through the one or more measurement channels using a second electrode of the wearable device, wherein determining the presence or the concentration of the one or more substances is based at least in part on the second signal.

6. The method of claim 1, further comprising:
generating an electrical current using an electrode of the wearable device, wherein the electrical current is configured to stimulate one or more sweat glands of the user, wherein collecting the bodily fluid is based at least in part on generating the electrical current.

7. The method of claim 1, further comprising:
causing a user device associated with the wearable device to display a message associated with the presence or the concentration of the one or more substances.

8. The method of claim 1, further comprising:
causing a user device associated with the wearable device to display instructions for the user to manipulate the fluid collection component to collect the bodily fluid, to manipulate the wearable device relative to the one or more measurement channels, or both, wherein collecting the bodily fluid, transmitting the light, generating the one or more signals, or any combination thereof, is based at least in part on the instructions.

9. The method of claim 1, wherein the one or more light-emitting components and the photodetector are configured to acquire physiological data from the user, the physiological data comprising photoplethysmogram (PPG) data, heart rate data, heart rate variability data (HRV), blood oxygen saturation data, or any combination thereof.

10. The method of claim 1, wherein the fluid collection component comprises an adhesive patch, a lateral flow patch, a microfluidic chamber, a material disposed on a surface of the wearable device, or any combination thereof.

11. The method of claim 1, wherein the fluid collection component comprises an attachment component configured to couple with the wearable device.

12. The method of claim 11, wherein the attachment component is configured to position the one or more measurement channels proximate to the one or more light-emitting components, the photodetector, or both.

13. The method of claim 1, wherein the bodily fluid comprises perspiration, saliva, tears, blood, or any combination thereof.

14. The method of claim 1, wherein the one or more substances comprise sodium chloride, cortisol, lactate, sodium, potassium, calcium, urea, alcohol, ammonium, glucose, inflammatory markers, toxic substances, or any combination thereof.

15. The method of claim 1, wherein the wearable device comprises a wearable ring device.

16. A system for fluid analysis, comprising:
a fluid collection component configured to collect a bodily fluid of a user, wherein the fluid collection component comprises one or more measurement channels configured to change color when exposed to the bodily fluid;
one or more light-emitting components of a wearable device;
a photodetector of the wearable device; and
a controller communicatively coupled with the one or more light-emitting components and the photodetector, the controller configured to:
transmit light associated with one or more wavelengths using the one or more light-emitting components;
generate, using the photodetector of the wearable device, one or more signals based at least in part on the light reflected off the one or more measurement channels of the fluid collection component and received by the photodetector;
determine a color of the one or more measurement channels based at least in part on the one or more signals; and
determine a presence or concentration of one or more substances within the bodily fluid based at least in part on the color.

17. The system of claim 16, wherein the controller is further configured to:
transmit first light associated with a first wavelength using the one or more light-emitting components;
generate a first signal based at least in part on the first light reflected off the one or more measurement channels and received by the photodetector;
transmit second light associated with a second wavelength using the one or more light-emitting components; and
generate a second signal based at least in part on the second light reflected off the one or more measurement channels and received by the photodetector, wherein determining the color is based at least in part on a comparison of the first signal and the second signal.

18. The system of claim 17, wherein the first wavelength or the second wavelength comprise a wavelength associated with infrared light.

19. The system of claim 16, wherein the one or more measurement channels comprise a reference channel that is configured to not change color when exposed to the bodily fluid, wherein the controller is further configured to:
generate one or more additional signals based at least in part on the light reflected off the reference channel and received by the photodetector, wherein determining the color of the one or more measurement channels is based at least in part on a comparison of the one or more signals and the one or more additional signals.

20. The system of claim 16, wherein the controller is further configured to:
generate an electrical current using a first electrode of the wearable device; and
generate a second signal based at least in part on the electrical current received through the one or more measurement channels using a second electrode of the wearable device, wherein determining the presence or the concentration of the one or more substances is based at least in part on the second signal.
